(19) United States
(12) Patent Application Publication
(10) Pub. No.: US 2020/0057057 A1
(43) Pub. Date: Feb. 20, 2020

(54) Methods and reagents for Toxoplasma infection diagnosis

(71) Applicants: Denis LIMONNE, Chicago, IL (US); Rima MCLEOD, Chicago, IL (US); Francois PEYRON, Chicago, IL (US); Kamal EL-BISSATI, Chicago, IL (US); Joe LYKINS, Chicago, IL (US)

(72) Inventors: Denis LIMONNE, Chicago, IL (US); Rima MCLEOD, Chicago, IL (US); Francois PEYRON, Chicago, IL (US); Kamal EL-BISSATI, Chicago, IL (US); Joe LYKINS, Chicago, IL (US)

(21) Appl. No.: 16/542,865

(22) Filed: Aug. 16, 2019

Related U.S. Application Data
(60) Provisional application No. 62/718,984, filed on Aug. 16, 2018.

(51) Int. Cl. G01N 33/543; G01N 33/569

(52) U.S. Cl. CPC G01N 33/54306; G01N 2800/26; G01N 2469/20; G01N 33/569

(57) ABSTRACT

Point-of-care (POC) testing for T. gondii infection can potentially address cost concerns and lead to better clinical outcomes through improved access to screening. This disclosure provides methods and compositions for whole blood POC testing to identify T. gondii infection with high sensitivity and specificity, obviating the need for venipuncture and sample processing infrastructure, making it an efficient, low-cost POC test.

### CROSS REFERENCE

This application claims priority to U.S. provisional patent application No. 62/718,984, filed Aug. 16, 2018, the disclosure of which is incorporated by reference herein in its entirety.

### GOVERNMENT SUPPORT

This invention was made with government support under AI027530 awarded by National Institutes of Health. The government has certain rights in the invention.

### BACKGROUND

It is now well established that monthly gestational serologic testing for acquisition of T. gondii infection for the first time during gestation facilitating prompt treatment can markedly improve patient outcomes and well-being. This can be utilized to permit prompt diagnosis and thereby treatment to prevent transmission to the fetus and also to ameliorate symptoms, resulting in saving life, cognition, motor function, sight, and dramatic benefit for improvement of quality of life. Toxoplasmosis causes substantial morbidity and mortality on a global scale. This disease can be severe. Vertical transmission from mother to fetus occurs from primary, acute infection during gestation. Congenital infection may result in chorioretinitis, hydrocephalus, epilepsy, and death. Serologic screening during gestation leads to early antenatal detection and rapid initiation of treatment with economic and patient outcome benefits. However, conventional serologic screening can be cost-prohibitive. Infrastructure, including electricity and equipment for sample processing, may be unavailable. Point-of-care (POC) tests detecting T. gondii infection offer solutions, potentially addressing cost concerns and leading to better clinical outcomes through improved access to screening. Provided herein, is proof of principle for potential utility and widespread applicability in clinical settings for a lateral flow immunochromatography-based Toxoplasma ICT IgG-IgM test using whole blood. This whole blood POC test performed with high sensitivity and specificity, obviating the need for venipuncture and sample processing infrastructure, making an efficient, low-cost POC test.

### SUMMARY OF THE INVENTION

The ability to provide test results rapidly to a subject in need thereof is very important to impact outcomes of multiple conditions. Rapid tests to aid diagnosis and enable early detection of multiple diseases and physiologic conditions are being developed. Such tests are especially useful when they can be applied with self-testing and require little in the way of laboratory processing. Examples of point-of-care (POC) test devices in common use today include pregnancy and fertility tests. Development of diagnostic tests for infections that use POC testing are especially important in resource-poor settings; for this reason, POC testing has become a new goal to be achieved for infections such as Toxoplasma infection.

The inventors have surprisingly discovered that a whole blood test (using only about 30 microliters) can perform with high sensitivity and specificity, obviating the need for venipuncture (since the whole blood can be obtained, for example, by finger prick) and sample processing infrastructure (required to separate serum, etc. from whole blood), making the methods as disclosed herein an extremely efficient, low-cost, point of care (POC) test for Toxoplasma infection and treatment monitoring. Such POC testing has potential to significantly expand access to screening, for example, during gestation for this serious, potentially fatal infection, with spillover benefit in facilitating screening programs for other congenital infections and improvements in maternal fetal health and well-being and care.

As provided herein, demonstration of the high performance of this whole-blood POC test, and the test's strong functionality at the point-of-care, which has not been previously demonstrated, provides the proof of principle of its potential utility and widespread applicability in clinical settings. This new test also fulfills the World Health Organization criteria for the ideal POC test (Affordable, Sensitive, Specific, User-friendly, Rapid/robust, Equipment-free, and Deliverable to users; A.S.S.U.R.E.D.). Performance and interpretation of this POC test takes less than two minutes of operator time, with results available to be interpreted within 20-30 minutes, facilitating appropriate clinical intervention so that it is ideal for following those who are seronegative to detect seroconversion.

In one aspect, the invention provides a method for diagnosing a Toxoplasma infection in a subject at risk of a Toxoplasma infection, comprising contacting a whole blood sample from the subject to a sample well of a lateral flow immunochromatography device to diagnose a likelihood of the subject having a Toxoplasma infection.

In certain embodiments of the method for diagnosing a Toxoplasma infection, the lateral flow immunochromatography device comprises:
(a) providing a cassette, wherein the cassette comprises:
   (i) a nitrocellulose strip comprising a Toxoplasma antigen immobilized on a test band; and
   (ii) a conjugate pad in fluid communication with the nitrocellulose strip impregnated with test latex particles coupled with a Toxoplasma antigen; and
   (iii) a sample well in fluid communication with the conjugate pad;
(b) adding the whole blood sample to the sample well to initiate migration of the whole blood and the test latex particles along the conjugate pad, under conditions to promote binding of anti-Toxoplasma antibodies in the whole blood sample to the Toxoplasma antigen on the test latex particles to form positive complexes; and
(c) detecting an interaction between the positive complexes and the test band.

In certain embodiments, approximately 30 μL of the whole blood sample is added to the sample well. In some embodiments, the nitrocellulose strip further comprises control latex particles that are optically distinguishable from the test latex particles, and wherein the conjugate pad of further comprises control antigens immobilized on a control band. In an embodiment, the test latex particles are black and the control latex particles are blue. In yet another embodiment, the control latex particles are impregnated with goat anti-rabbit antibodies, and wherein the control antigens comprise rabbit gamma immunoglobulins. In some embodiments, the anti-Toxoplasma antibodies in the whole blood sample are IgG and/or IgM antibodies.

In certain embodiments, the methods as disclosed herein comprise dispensing an eluting solution to the sample well, subsequent to the whole blood sample, and allowing migration of the whole blood sample and the eluting solution for 20-30 minutes before reading the test band and the control band of the device.

In certain embodiments, the Toxoplasma infection is the result of an infection by Toxoplasma gondii. In some embodiments, the subject is a pregnant human or a human suspected of being pregnant.

In certain embodiments, the method further comprises treating those subjects that are identified as likely to have a Toxoplasma infection with a therapeutic for treating Toxoplasma infection. In some embodiments, the therapeutic is selected from the group consisting of: pyrimethamine, sulfadiazine, sulfapyrizine, sulfamethazine, sulfamerizine, azithromycin, clarithromycin, atovaquone, dapsone, cotrimoxazole, and combinations thereof.

In a second aspect, the invention provides a kit comprising a lateral flow immunochromatography device for diagnosing a Toxoplasma infection in a subject wherein the kit comprises:
(a) the lateral flow immunochromatography device, wherein the device comprises a cassette comprising:
   (i) a nitrocellulose strip on which are immobilized a Toxoplasma antigen in a test band and a control antigen in a control band;
   (ii) a conjugate pad in fluid communication with the nitrocellulose strip impregnated with test latex particles coupled with a Toxoplasma antigen and control latex particles coupled with a control antigen; and
   (iii) a sample well in fluid communication with the conjugate pad;
(b) eluting buffer.

In certain embodiments of the kit, the eluting buffer is in a 3 mL dropper bottle.

In some embodiments, the kit comprises: 60 lateral flow immunochromatography devices, wherein there are 6 bags, and each bag contain 10 devices and a desiccant packet; 3 dropper bottles with the eluting buffer, where each dropper bottle comprises 3 mL of eluting buffer; and instructions for use. In yet another embodiment, the kit comprises: lancets for fingertip whole blood sampling; capillary tubes for collecting the whole blood sample; and a timer.

The methods and kits described herein provide for more effective diagnosis and/or treatment of life-threatening or irreversibly debilitating human disease or conditions resulting from infection from T. gondii. The methods and kits described are being considered for the FDA Breakthrough Devices Program and meet the WHO ASSURED criteria. Currently, there are no approved or cleared alternatives for whole blood testing from a finger stick (i.e. using ˜30 μL of whole blood), and the methods and kits as described herein provide significant advantages over existing approved and/or cleared alternatives. Finally, the methods and kits as described herein are in the best interest of the patients.

### BRIEF DESCRIPTION OF THE DRAWINGS

FIG. 1A: is a schematic of the study participant composition in United States and Morocco. One seropositive individual was tested via point-of-care (POC) test twice, but is included in this number only once. Nineteen (19) pregnant women were tested a total of three times each as part of a pilot gestational screening program. All were seronegative. Volunteers learned of the study through word of mouth sometimes when friends or family members knew of an affected child and asked to participate or were formally recruited in an obstetrical practice. No one complained or mentioned medical problems of any type other than as related to toxoplasmosis among those who were seropositive from the NCCCTS or pregnancy, although there was no health questionnaire or physical examination as part of this study.

FIG. 1B: Performing the Toxoplasma ICT IgG+IgM Test. The site of the finger-stick is cleansed with an alcohol wipe and pricked with a lancet. Blood is collected via capillary tube and applied to the test kit. Four drops of eluent are then applied. The test can be interpreted in 20 to 30 minutes. The black arrow adjacent to the ‘T’ indicates the presence of antibodies against T. gondii. NCCCTS, National Collaborative Chicago-Based Congenital Toxoplasmosis Study; POC, point of care; UofC, University of Chicago.

FIG. 2: Shows an exemplary device with an immunochromatographic test strip included in a cassette. From left to right, the strip is composed of a cellulose pad called “sample pad” (1) where are dispensed the sample and the eluent in the “sample well” (2). Then there is a fiberglass pad, the “conjugate pad” (3) loaded with blue latex particles coated with anti-rabbit goat antibodies and black latex particles coated with toxoplasma antigens (obtained from in vivo culture). After the conjugate pad, there is a nitrocellulose sheet (4) on which are dispensed two lines of reagent, one of toxoplasma antigens, the “T”, “test line” (5) and one of rabbit antibodies the “C”, “control line” (6). If anti-toxoplasma antibodies are present in the sample, the test line (“T”) will appear in black. The control line (“C”) will appear regardless of the presence of anti-toxoplasma antibodies in the sample. The absence of the control line demonstrates the failure of the test and results can't be interpreted. Finally, a second cellulose pad at the right-end, the “absorbent pad” (7) completes the strip.

FIG. 3A: Shows exemplary devices showing both negative (left) and positive (right) results.

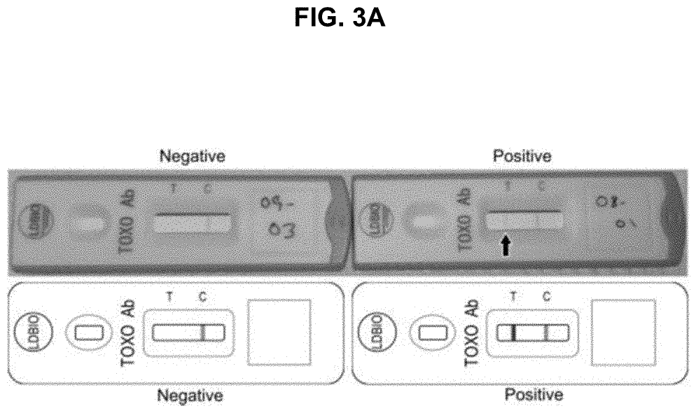

FIG. 3B: Shows exemplary devices showing both negative (top) and positive (bottom) results that could be included, for example, in training and instruction materials with the device.

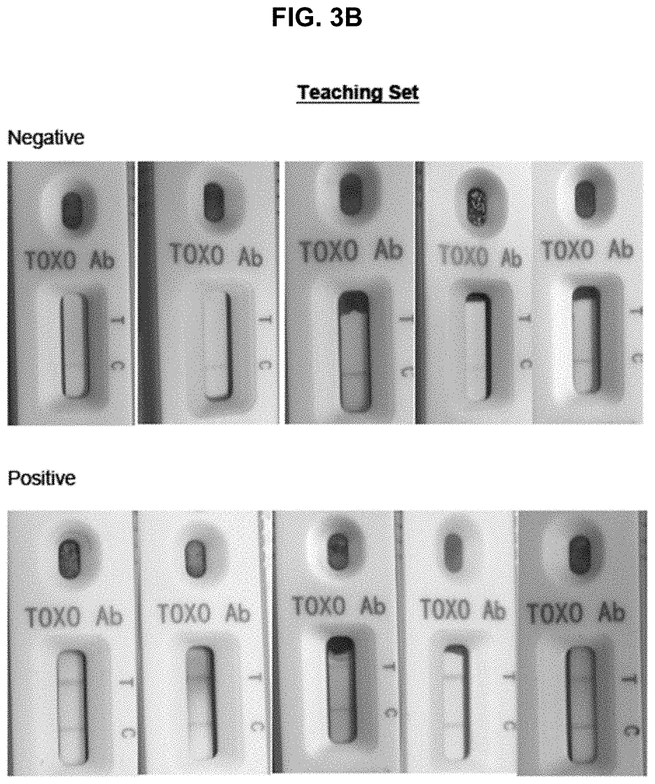

### DETAILED DESCRIPTION OF THE INVENTION

All references cited are herein incorporated by reference in their entirety.

Terms used in the claims and specification are defined as set forth below unless otherwise specified. In the case of direct conflict with a term used in a parent provisional patent application, the term used in the instant specification shall control.

As used herein, the singular forms “a”, “an” and “the” include plural referents unless the context clearly dictates otherwise. “And” as used herein is interchangeably used with “or” unless expressly stated otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

All embodiments disclosed herein can be used in combination, unless the context clearly dictates otherwise.

In one aspect, this disclosure provides methods for diagnosing a Toxoplasma infection in a subject at risk of a Toxoplasma infection, comprising contacting a whole blood sample from the subject to a sample well of a lateral flow immunochromatography device to diagnose a likelihood of the subject having a Toxoplasma infection.

In certain embodiments, the method is used to diagnose whether the subject is suffering from a T. gondii infection. In such an embodiment, the subject is suspected of suffering from a T. gondii infection based on the presence of one or more symptoms, and the methods can be used to assist in providing a more definitive diagnostic, along with all other factors to be considered by attending medical personnel. The methods may be used to determine infection by any wild type, mutant, or recombinant Toxoplasma strain. In an embodiment, the Toxoplasma comprises any wild type, mutant, or recombinant Toxoplasma gondii strain. For example, the methods and kits as disclosed herein may be used to determine infection by genetically polymorphic strains from Argentina, Austria, Colombia, France, Germany, Lithuania, Morocco, Panama, Uruguay, and/or the United States. Additionally, in some embodiments, the methods and kits provided herein can be used to detect an acute infection. In certain embodiments, the methods and kits provided herein can be used to detect a chronic infection.

In certain embodiments of the methods disclosed herein, the lateral flow immunochromatography device comprises:
(a) providing a cassette, wherein the cassette comprises:
   (i) a nitrocellulose strip comprising a Toxoplasma antigen immobilized on a test band; and
   (ii) a conjugate pad in fluid communication with the nitrocellulose strip impregnated with test latex particles coupled with a Toxoplasma antigen; and
   (iii) a sample well in fluid communication with the conjugate pad;
(b) adding the whole blood sample to the sample well to initiate migration of the whole blood and the test latex particles along the conjugate pad, under conditions to promote binding of anti-Toxoplasma antibodies in the whole blood sample to the Toxoplasma antigen on the test latex particles to form positive complexes; and
(c) detecting an interaction between the positive complexes and the test band.

In one embodiment, the test is run by successively dispensing the whole blood sample and an eluting solution (called the eluent) in the “sample well” of the cassette. Adding the eluent starts the concomitant migration (chromatography) of the whole blood sample and the test latex particles and the control latex particles. This migration is completed in 20-30 minutes. If anti-Toxoplasma antibodies (IgG and/or IgM) are present in the whole blood sample, then a complex is formed between the test latex particles coupled to toxoplasma antigens and the patient's antibodies which is then captured by the test band. For detection, the latex test particles may be colored (such as black), in which case results in the appearance of a black line at the T-band if the test is positive. The control latex particles may be optically distinguishable from the test latex particles (for example, blue colored latex particles) such that direct capture of the control latex particles by the control antigens on the nitrocellulose strip C-band results in the appearance of a blue line, meaning that the chromatography performed well. The binding of black latex particles to the test line is done by the bivalent property of antibodies (or pentavalence of IgM), as the same antigen is used in both the coating of latex test particles and the test line. In some embodiments, the methods and kits provided herein can be used to detect false positive IgM antibodies from the subject. The binding of blue latex particles to the control line is done by the direct antibody-antigen reaction between the anti-rabbit goat antibodies (that act as antibodies) and the rabbit antibodies (that act as antigens).

As used herein, the term “subject” refers to any animal, including mammals, but not limited to, mice, rats, other rodents, rabbits, dogs, cats, swine, cattle, sheep, horses, or primates, and humans. In some embodiments, the subject is pregnant. In certain embodiments, the subject is a human. In an embodiment, the subject is a pregnant human. In certain embodiments, the pregnant human is tested within the first two weeks of pregnancy. In some embodiments, the pregnant human is tested before the third week of pregnancy. In some embodiments, the pregnant human is tested before the fourth week of pregnancy. In some embodiments, the pregnant human is tested before the fifth week of pregnancy. In some embodiments, the pregnant human is tested before the sixth week of pregnancy. In some embodiments, the pregnant human is tested before the seventh week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the tenth week of pregnancy. In some embodiments, the pregnant human is tested before the twelfth week of pregnancy. In some embodiments, the pregnant human is tested before the fourteenth week of pregnancy. In some embodiments, the pregnant human is tested before the sixteenth week of pregnancy. In some embodiments, the pregnant human is tested before the eighteenth week of pregnancy. In some embodiments, the pregnant human is tested before the twentieth week of pregnancy. In some embodiments, the pregnant human is tested before the twenty-second week of pregnancy. In some embodiments, the pregnant human is tested before the twenty-fourth week of pregnancy. In some embodiments, the pregnant human is tested before the twenty-sixth week of pregnancy. In some embodiments, the pregnant human is tested before the twenty-eighth week of pregnancy. In some embodiments, the pregnant human is tested before the thirtieth week of pregnancy. In some embodiments, the pregnant human is tested before the thirty-second week of pregnancy. In some embodiments, the pregnant human is tested before the thirty-fourth week of pregnancy. In some embodiments, the pregnant human is tested before the thirty-sixth week of pregnancy. In some embodiments, the pregnant human is tested before the thirty-eighth week of pregnancy. In some embodiments, the pregnant human is tested before the fortieth week of pregnancy. In an embodiment of the method, the pregnant human is tested by week eight of the pregnancy.

Suitable whole blood samples can be in liquid form to allow interaction with the sample well of the device. In certain embodiments, only approximately twenty to thirty microliters (˜20-30 μL) is added to the sample well. In certain embodiments, approximately twenty microliters (˜20 μL) is added to the sample well. In certain embodiments, approximately twenty-five microliters (˜25 μL) is added to the sample well. In certain embodiments, approximately 30 microliters (˜30 μL) is added to the sample well. As only approximately twenty to thirty microliters (˜20-30 μL) is required for the methods as disclosed herein, a simple finger stick can provide more than enough whole blood, obviating the need for venipuncture (since the whole blood can be obtained, for example, by finger stick) and sample processing infrastructure (required to separate serum from whole blood), making the methods and kits as disclosed herein an extremely efficient, low-cost, point-of-care (POC) test for Toxoplasma infection. Lancets can be used in combination with capillary tubes to collect and dispense the whole blood sample. Lancets of varying depths can be used, depending upon the desired blood flow and patient age and weight. When the tip of a capillary tube touches a whole blood droplet drawn by a lancet, blood will flow into the capillary tube via capillary action. After whole blood collection, pressure can be applied to the puncture site with a dry sterile swab until bleeding stops. If an infant's heel was punctured, then the foot should be elevated above the body. Once clotting has occurred an adhesive bandage can be applied if desired.

In certain embodiments, a nitrocellulose strip is used in the lateral flow immunochromatography device. Nitrocellulose membrane layers for lateral flow assays are well-known in the art and are composed of interconnected nitrocellulose fibers. There are multiple benefits to using nitrocellulose for the primary membrane: low cost, capillary flow high affinity for protein biding, and ease of handling. Nitrocellulose has high protein binding. In other embodiments, any substrate capable of providing liquid flow for lateral flow membranes can be used. Such materials are known in the art and include nylon, cellulose acetate, glass fibers, cross-linked dextran and other porous polymers.

In certain embodiments, the nitrocellulose strip comprises a test band (T-band) or test line (T line) and a control band (C-band) or control line (C line). In an embodiment, the test line comprises toxoplasma antigens. Thus, if anti-toxoplasma antibodies are present in the whole blood sample from the subject, then the anti-toxoplasma antibodies in the whole blood sample will bind the toxoplasma antigens coupled to the test latex particles and to the toxoplasma antigens in the test line and the test line (“T”) will appear in black. The control line (“C”) will appear regardless of the presence of anti-toxoplasma antibodies in the sample. The absence of this control line demonstrates the failure of the test and results can't be interpreted.

As used herein, the term “conjugate pad” refers to an area the whole blood first moves through after the sample well and comprises a moveable conjugate of a detectable marker (i.e. a black latex particle conjugated to a toxoplasma antigen). In certain embodiments, the conjugate pad is a fiberglass pad. When the whole blood to be analyzed flows from the sample well through the conjugate pad, the conjugate (test latex particle coupled to a toxoplasma antigen) binds to the anti-toxoplasma antibody present in the whole blood sample, and the complex flows with the whole blood sample to the nitrocellulose strip. Thus, in certain embodiments, the device comprises a conjugate pad in fluid communication with the nitrocellulose strip. In the nitrocellulose strip, the ligand-conjugate complex binds to immobilized binding agent (i.e. toxoplasma antigen). In some embodiments, the conjugate pad comprises test latex particles coupled with a toxoplasma antigen. In certain embodiments, the test latex particles are black. In some embodiments, the conjugate pad comprises control latex particles coupled with control antigens. In certain embodiments, the control latex particles are blue.

As used herein, the terms “sample well” or “sample pad” refer to the area of the device where the whole blood sample and the eluting buffer are first dispensed on the device. In certain embodiments, the sample pad of the fluid cassette is in fluid contact with the conjugate pad of the cassette. In some embodiments, the sample pad is a cellulose pad.

Any suitable control can be used, including but not limited to a reference value obtained from one or more subjects that either do not have a T.gondii infection, or that are known to have a T. gondii infection, a previous blood sample obtained from the same subject, or any other suitable control. It is well within the level of those of skill in the art to determine an appropriate control for an intended use in light of the teachings herein. The change in level from control that correlates with T. gondii infection in the subject may be a difference of 10%, 25%, 50%, 75%, 100%, or more. In an embodiment, the difference is a statistically significant increase as judged by standard statistical analysis.

In certain embodiments, the method further comprises treating those subjects that are identified as likely to have a Toxoplasma infection with a therapeutic for treating Toxoplasma infection. In some embodiments of the method, the subject has toxoplasmosis, and the treating comprises reducing severity of one or more symptoms of toxoplasmosis, and/or reducing recurrence of symptoms of toxoplasmosis. In some embodiments of the method, the treating comprises reducing parasitic load in the subject. In some embodiments of the method, the treating comprises reducing the bradyzoite form and/or the tachyzoite form of the parasite in the subject. In some embodiments of the method, the method further comprises administering to the subject one or more additional compounds in an amount effective to treat the infection. In some embodiments of the method, the one or more additional compounds are selected from the group consisting of pyrimethamine, sulfadiazine, cycloguanil, inhibitors of calcium kinases or dense granules or vacuolar atpases, atovoquone, and bulky cytochrome Qi inhibitors, itraconazole and other inhibitors of T. gondii. Therapies may include, but are not limited to pyrimethamine, sulfadiazine, sulfapyrizine, sulfamethazine, sulfamerizine, azithromycin, clarithromycin, atovaquone, dapsone, cotrimoxazole, and combinations thereof (also see WO2017/112678, which is incorporated by reference herein in its entirety). In a further embodiment, the methods may comprise modifying a course of treatment if the subject is not responding appropriately, as determined by attending medical personnel.

In another aspect, the disclosure provides a kit comprising the lateral flow immunochromatography device as disclosed herein. The assembly of the kit can be packaged for use at a single facility where individual components can be grouped to make its use efficient, self-contained and disposable as self-contained for blood borne pathogens or for single use by patient. In the later, in a single small container the following disposable materials are placed: non-breakable capillary tube, finger prick device (e.g. lancet), alcohol wipes, timer for 20-30 minute time alert for reading, link to educational materials, and instructions for use.

In an embodiment, a kit can comprise components for 60 ready to use tests. In such an embodiment, the kit comprises 6 bags (sealed and with zip closure) of 10 devices including a desiccant packet, 3×3 ml of the eluent (dropper bottle), and instructions for use.

Cassettes can be packed in sealed aluminum, PE, or PET bags with a silica gel bag (desiccant). The use of such desiccants is intended to avoid humidity and condensation, due to the very low air permeability of the sealed bag and the silica gel that can capture any residual humidity. The eluting solution is stored in plastic dropper. All batches of dropper are controlled before use to ensure that they do not present a risk of leakage.

In some embodiments, the kit can comprise additional materials, for example, such as a micropipette and disposable tips for dispensing volumes of 15 to 45 μL, and a timer. In certain embodiments, additional materials can include capillary tubes and lancets for fingertip whole blood sampling. In yet additional embodiments, the kits can comprise supplies for general safety precautions, such as gloves and appropriate apparel for protection against exposure to blood borne pathogens. Dry sterile gauze and/or adhesive bandages can also be included in the kits as disclosed herein.

Each kit should be stored in the original sealed bag between 2 and 8° C. (do not freeze). Cassettes can be used until the expiry date written on the bag label. Kits and enclosed devices should not be used after the expiration date. Bags comprising the tests should be allowed to reach room temperature before opening in order to avoid condensation in the bag (e.g. allow at least 15 minutes for the bag to reach room temperature). Keep the bag, after opening at room temperature (18-30° C.), and carefully close (zip closure), with the desiccant packet inside. After opening, the cassettes can be used for up to 2 months. The eluting solution (eluent) is stable up to 2 months at room temperature (18-30° C.) and until expiration date (as written on the kit) if kept between 2 and 8° C.

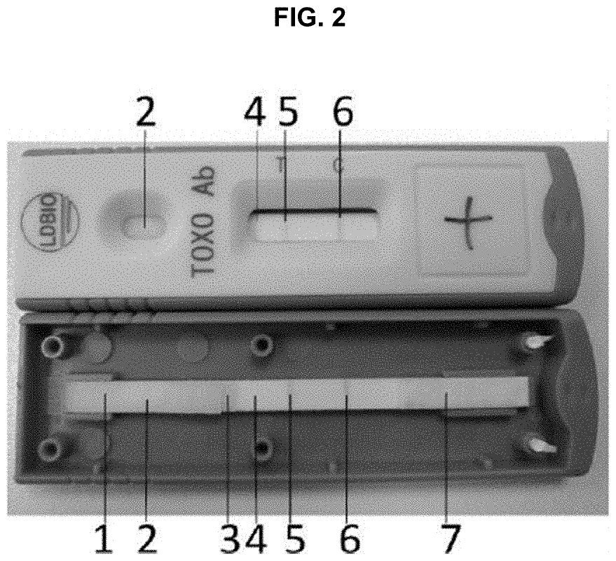

In yet another aspect, the disclosure provides a device. In certain embodiments, the device can be a lateral flow immunochromatographic test strip included in a cassette, which can be composed of a succession of pads allowing a continuous migration of a liquid from one end to the other (chromatography), as shown in FIG. 2. Inside the cassette, the device is composed of: a nitrocellulose strip on which are spread two reactive bands: the antigens (Toxoplasma gondii) of the “test” band (T band) and the rabbit gamma globulins of the “control” band (C band), and a fiberglass support (conjugate pad) which is impregnated of black latex particles coupled with Toxoplasma gondii antigens (“test” latex=T latex) and blue latex particles coupled with goat anti-rabbit IgG (“control” latex=C latex). The test is run by successively dispensing the whole blood sample and an eluting solution (called the eluent) in the sample well of the cassette. The liquid eluent solution, can be composed of solvents (<5% polar ionic solvents) and detergents (<1%), and ensures the chromatographic migration of the sample. Its composition is designed to optimize sensitivity and specificity of the test. Adding the eluent starts the concomitant migration (chromatography) of the serum and the latex particles. This migration is completed in about 20-30 minutes. If specific anti-toxoplasma antibodies (IgG and/or IgM) are present in the whole blood sample, then a complex is formed between the test latex particles and the patient's antibodies which is then captured by the test band, which results in the appearance of a black line at the T band (i.e., the test is positive). The direct capture of the control latex particles by the C band results in the appearance of a blue line, meaning that the chromatography performed well. The appearance of this blue line is systematic and independent of the anti-toxoplasma antibody status of the patient. Both letters “T” and “C” can be printed on the cassette, materializing the position of the corresponding reading area.

From left to right (see FIG. 2), the strip can be composed of a cellulose pad called “sample pad” (1) where are dispensed the whole blood sample and the eluent in the “sample well” (2). Then there is a fiberglass pad, called the “conjugate pad” (3) loaded with blue latex particles coated with anti-rabbit goat antibodies and black latex particles coated with toxoplasma antigens (obtained from in vivo culture). After the conjugate pad, there is a nitrocellulose sheet (4) on which are dispensed two lines of reagent, one of toxoplasma antigens, the “test line” (5) and one of rabbit antibodies the “control line” (6). If anti-toxoplasma antibodies are present in the whole blood sample, the test line (“T”) will appear in black. The control line (“C”) will appear regardless of the presence of anti-toxoplasma antibodies in the sample. The absence of this blue control line demonstrates the failure of the test and results can't be interpreted. Finally, a second cellulose pad at the right-end, the “absorbent pad” (7) completes the strip (see FIG. 2).

The binding of black latex particles to the test line is done by the bivalent property of antibodies (or pentavalence of IgM), as the same antigen is used in both the coating of latex test particles and the test line. The binding of blue latex particles to the control line is done by the direct antibody-antigen reaction between the anti-rabbit goat antibodies (that act as antibodies) and the rabbit antibodies (that act as antigens).

Quality control of the test can be monitored and ensured as follows:
Batch certificate of conformity (homogeneity, analytical performances, stability).
Quality insurance at production and control site (ISO 13189:2016 certified)
Presence of a control line for each test. In the absence, the test must be considered invalid.
Daily/Weekly routine control of a positive and negative control samples, on the same batch number as the one used with patients.

### Reading and Interpretation

An exemplary procedure for the methods, and/or kits and devices as disclosed herein is provided here. Administer approximately 30 μl of whole blood in the sample well, and then dispense 4 drops of the eluent, keeping the dropper vertical while dispensing. Do not use the eluent of another lot number. Close the dropper after use. Start the timer. The reading must be done near a window or under direct light (example: a desk lamp), and avoid shadows on the reading area. The reading must be done between 20 and 30 minutes after starting the timer, and do not take into account the results from readings after 30 minutes. A positive test is 2 lines, a black “T” line and a blue “C” line appear in the corresponding areas. Every “T” line must be considered positive, even of very weak intensity. For very weak lines, make the reading with the eye vertically above the reading area. For a negative test: no black line appears. Only the blue “C” line is visible. For an equivocal test: in very rare cases, a faint, diffuse, grey line can appear on the “T” band. This result should be considered negative, but controlled on another sample or technique. For an invalid test: the “C” line does not appear. Read once again the instructions and repeat the test. This is a qualitative test. Intensity of the black line does not reflect the quantity of anti-Toxoplasma antibodies in the sample. Positivity of the test is proof of the contact of the patient with the infectious agent, but doesn't prejudge the contact date or the clinical status of the patient. For quality control, the blue “C” line allows the validation of the good running of the test. However it is recommended to incorporate from time to time a known weak positive sample in a run. The positivity can be caused by the presence of IgG and/or IgM directed against the infectious agent, the test does not distinguish the type of antibodies present.

### Manufacturing Process

All steps are guided by procedures and forms, and performed by trained technicians, with a bachelor degree in life science or equivalent and trained to the procedure. Forms are designed to ensure traceability of all steps of manufacturing process and batch number and quantity of materials used. Time spent on all incubation steps is also recorded.

The first step of a production is to purify antigen obtained from in vivo culture of Toxoplasma gondii. At the end of purification, antigen concentration is adjusted by 280 OD to a desired level. The purified antigen is then coupled on black latex particles, using a crosslinker. The coupled latex particles are then distributed on glassfiber papersheet that are then dried then cut in strips 30 cm long. Purified antigen is also dispensed on 30 cm nitrocellulose cards by a dedicated automated dispenser. Finally nitrocellulose cards, glassfiber strips and two cellulose pads (one sample pad and one absorbent pad) are aggregated together then cut in 3.9 mm width strips. Those strips are then put into the plastic frame.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### EXAMPLES

### Methods:

At each monthly obstetrical visit a finger stick sample with 30 microliter of whole blood was collected following cleansing the finger tip with an alcohol wipe and applying pressure so the fingertip was suffused with blood. The 30 microliters of blood, half the height of the capillary tube, was drawn into the capillary tube. The blood in the capillary tube was applied to the sample well in the lateral flow immunochromatography device, and 4 drops of the eluting solution (eluent) was added. This was performed by a nurse who then cleaned the finger again with an alcohol wipe and applied a small bandage. The medial lateral aspect of the fingertip was used as it is generally less calloused. The lateral flow immunochromatography cassette was placed in a small plastic bag with a timer attached and brought to the appointment with the physician with the timer set for 20 minutes. Two movies were provided to illustrate its use as well as the photographs in the manuscripts provided with the kit. The obstetrician or nurse midwife and his or her nurse read the result, photographed it with a camera and sent it to the central monitor and to the Epic chart in the site at The University of Chicago. Two independent readers documented their observations and there was a photographic record. This can also be performed by heel stick for an infant or with whole blood obtained by phlebotomy in certain circumstances where rapid diagnosis was important. Any such test would always have standard of care test backup so this study would be exempt.

Back up testing was with serum by using either Sabin Feldman dye test/IgM ELISA or Abbott Architect IgG and IgM performed in USA and French reference laboratories. There was 100% concordance when it was tested in the USA in research studies. It was introduced into the clinical setting where it also performed extremely well. The POC test as described herein, has met all the WHO criteria for an ideal POC test including high performance, sensitive, specific, easy to use, rapid, low technology, and inexpensive. It outperformed two other POC tests one made in the US, and one made in Ireland. The lateral flow immunochromatography device also detects false positive IgM results in standard tests used commercially in the US.

### Volunteer Recruitment and Obtaining Samples

Samples were obtained from consenting volunteers, in the U.S. including seropositive individuals affiliated with the NCCCTS and obstetrical patients in Chicago, and obstetrical patients in Morocco (Table 1A-1C). No incentives were provided for participation. Each person of unknown serologic status underwent venipuncture, and status was confirmed either with ARCHITECT Toxo-IgG and IgM system in the Lyon, France Reference Laboratory (n=95 persons), or for Moroccan patients, with Platelia™ Toxo IgG and IgM system (n=39).

TABLE 1A
Methods, Test Results, and Parameters for Sensitivity
and Specificity and Confidence Intervals

| | | | Serology | Serum & POC | United | |
|---|---|---|---|---|---|---|
| | | | Known | Tested | States | Morocco |
| | Patients | Samples | Already | Concurrently | Patients | Patients |
| Seronegative * | 105 | 143** | 8 | 135 | 99 | 6 |
| (IgG and IgM | | | | | | |
| negative in | | | | | | |
| Reference tests) | | | | | | |
| Seropositive * | 100 | 101*** | 64 | 37 | 67 | 33 |
| (IgG and/or | | | | | | |
| IgM positive in | | | | | | |
| Reference tests) | | | | | | |

* For sera samples, pink-line and black-line POC tests were 100% concordant. For whole blood samples, it was determined that the pink-line test kit was not useful when testing whole blood, as the lighter indicator was obscured by the whole blood
**19 pregnant women were tested a total of 3 times each in an ongoing pilot program for screening during gestation
***1 person was tested twice to confirm accuracy of test across and at differing times

TABLE 1B
Totals by Country

| | Seronegative | Seropositive | Acute Samples |
|---|---|---|---|
| | Samples Tested | Samples Tested | (IgG + IgM |
| | (by POC and | (by POC and | Tested with |
| Country | Reference Test) | Reference Test) | Reference Test) |
| United States | 137 | 68 | 3^{#} |
| Morocco | 6 | 33 | 2^{#} |
| Total | 143 | 101 | 5^{#} |

^{#}Also included in seropositive person totals. The U.S. persons with IgM antibodies were sometimes followed over time, and all had adjunctive testing such as anti-T. gondii IgA, differential agglutination and avidity tests [2].

TABLE 1C
Test Results, Sensitivity, Specificity, Confidence Intervals

| | Seropositive | Seronegative |
|---|---|---|
| Test Result | (Reference Test) | (Reference Test) |
| Seropositive | 101⁺ | 0 |
| (Whole Blood- POC Test) | | |
| Seronegative | 0⁺⁺ | 143 |
| (Whole Blood- POC Test) | | |

| Test Parameter | Diagnostic Performance | 95% Confidence Interval |
|---|---|---|
| Sensitivity | 100% | 96.41-100%⁺⁺ |
| Specificity | 100% | 97.45-100%⁺⁺ |

⁺Confirmatory tests allow a provider to distinguish acute from chronic infection. Acute infection during gestation requires anti-Toxoplasma medicines to prevent or reduce vertical transmission. Chronic infection requires no further testing during gestation. The first test should be performed by 12 weeks gestation to facilitate distinction of acute and chronic infection [1].
⁺⁺A very faint grey Toxoplasma (T) band was noted transiently for one person tested prospectively. This band was not visible when photographed at 20 and 30 minutes. In accordance with the manufacturer's instructions, this result was designated “equivocal or indeterminate.” Any such equivocal result requires back up testing, as does any first positive result for a pregnant woman.

### Fingerstick Protocol, Whole Blood-POC Test Kit, and Comparison to Serum-Variant Test Kit

Participants provided whole blood via fingerstick. Participants' fingers were compressed, suffusing the tip, and cleaned with an alcohol wipe. A standard lancet was used for fingerstick. Capillary tubes allowed collection of 30 μL of blood, which was directly applied to the lateral flow immunochromatography device, followed by application of 4 drops of buffer, provided in the kit. Tests were interpreted at 20-30 minutes by the individual performing tests and photographed for later interpretation by two individuals unaware of the subjects' identity and serologic status.

### Ethics

All participants provided written, informed consent. This study was performed in accordance with rules and regulations of the University of Chicago Institutional Review Board under protocol #8793, and/or IRB-approval in Morocco.

### Results:

### Obtaining Samples

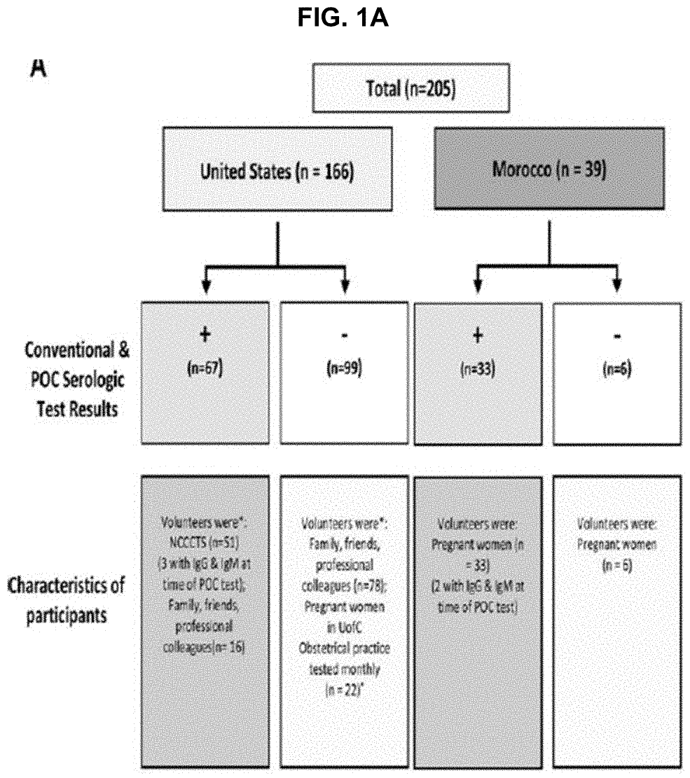

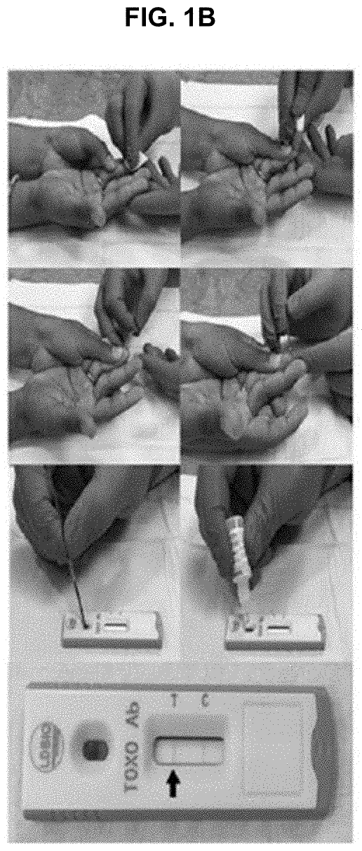

A total of 205 persons (FIG. 1A) (244 samples) had their serologic status for T. gondii assessed using the whole blood test and confirmed using their NCCCTS records (n=71), or concurrent standard laboratory testing (n=134). Nineteen seronegative pregnant women had fingerstick and venipuncture performed each month for the first three months, and three for the first month (two with pregnancy loss) in an ongoing pilot gestational screening program, adding 41 samples, making 244 total samples. Overall, 101 samples proved seropositive, including five from acutely infected individuals, who had IgM/IgG antibodies against T. gondii, while 143 were seronegative. Seropositive is defined here as having detectable anti-Toxoplasma IgG and/or IgM. For a small subset of pediatric patients (n=7, ages 3 weeks to 7 years) who were undergoing venipuncture as part of routine clinical care, whole blood was obtained from the needle tip to avoid a second, potentially distressing fingerstick.

### Sensitivity and Specificity of Whole Blood and Comparability to Serum-Variant Toxoplasma ICT IgG-IgM Test Kit

The whole blood test proved highly sensitive and specific, with sensitivity of 100% (95% CI: 96.41-100%) and specificity of 100% (95% CI: 97.45-100%) (Table 1A-1C). Whole blood, serum-variant, and reference testing demonstrated 100% concordance. Of note, individuals with lower levels of anti-Toxoplasma antibodies infected at remote times and with lower titers were positive in the POC test in the range detected in gold-standard tests.

### Viability of Fingerstick as Testing Modality

No participant refused a second fingerstick, although they were informed they could. Participants did not report any significant discomfort associated with fingerstick. Patients and providers enthusiastically accepted the monthly gestational screening program.

### Confirmatory Data

An additional 86 samples were tested for T. gondii using the methods and whole blood-POC test as disclosed herein, and these 86 samples were also assessed using concurrent standard ELISA laboratory testing. Overall, 18 samples proved seropositive, and of the 86 samples tested, there was only 1 discrepancy between the two tests (see Table 2).

The inventors have tested 64 additional people, 43 who were known to be seropositive for T. gondii infection and all were found to be positive in the whole-blood POC test; 21 people were negative in the whole-blood POC test, and were confirmed to be negative by a second standard predicate method (sensitivity 100%, specificity 100%; data not shown).

Additionally, 20 women were tested monthly through 9 months of gestation. The testing consistently showed 100% sensitivity and specificity (data not shown).

TABLE 2
Summary of confirmatory data of whole blood POC test.

| Sample | Whole-blood | |
|---|---|---|
| Number | POC test result | ELISA test result |
| 1 | Negative | Negative |
| 2 | Negative | Negative |
| 3 | Negative | Negative |
| 4 | Negative | Negative |
| 5 | Positive | 70 |
| 6 | Negative | Negative |
| 7 | Negative | Negative |
| 8 | Negative | Negative |
| 9 | Negative | Negative |
| 10 | Negative | Negative |
| 11 | Negative | Negative |
| 12 | Negative | Negative |
| 13 | Negative | Negative |
| 14 | Negative | Negative |
| 15 | Negative | Negative |
| 16 | Negative | Negative |
| 17 | Positive | 60 |
| 18 | Negative | Negative |
| 19 | Negative | Negative |
| 20 | Negative | Negative |
| 21 | Negative | Negative |
| 22 | Negative | Negative |
| 23 | Positive | 48 |
| 24 | Negative | Negative |
| 25 | Negative | Negative |
| 26 | Negative | Negative |
| 27 | Negative | Negative |
| 28 | Positive | 48 |
| 29 | Negative | Negative |
| 30 | Positive | 48 |
| 31 | Negative | Negative |
| 32 | Negative | Negative |
| 33 | Negative | Negative |
| 34 | Positive | Negative |
| 35 | Positive | >240 |
| 36 | Negative | Negative |
| 37 | Negative | Negative |
| 38 | Positive | 60 |
| 39 | Negative | Negative |
| 40 | Negative | Negative |
| 41 | Negative | Negative |
| 42 | Negative | Negative |
| 43 | Negative | Negative |
| 44 | Negative | Negative |
| 45 | Negative | Negative |
| 46 | Positive | * |
| 47 | Positive | * |
| 48 | Negative | Negative |
| 49 | Negative | Negative |
| 50 | Negative | Negative |
| 51 | Negative | Negative |
| 52 | Negative | Negative |
| 53 | Positive | 82 |
| 54 | Positive | 90 |
| 55 | Negative | Negative |
| 56 | Positive | 240 |
| 57 | Positive | 194 |
| 58 | Negative | Negative |
| 59 | Negative | Negative |
| 60 | Negative | Negative |
| 61 | Negative | Negative |
| 62 | Negative | Negative |
| 63 | Negative | Negative |
| 64 | Negative | Negative |
| 65 | Negative | Negative |
| 66 | Negative | Negative |
| 67 | Negative | Negative |
| 68 | Negative | Negative |
| 69 | Negative | Negative |
| 70 | Negative | Negative |
| 71 | Negative | Negative |
| 72 | Negative | Negative |
| 73 | Negative | Negative |
| 74 | Negative | Negative |
| 75 | Negative | Negative |
| 76 | Negative | Negative |
| 77 | Positive | >240 |
| 78 | Negative | Negative |
| 79 | Negative | Negative |
| 80 | Negative | Negative |
| 81 | Positive | 158 |
| 82 | Negative | Negative |
| 83 | Negative | Negative |
| 84 | Positive | 194 |
| 85 | Negative | Negative |
| 86 | Positive | 110 |

* confirmed positive with BioRad serology test

### Discussion:

Point-of-care testing for Toxoplasma infection has potential to markedly change clinical approach to this infection by detecting seroconversion, using small volumes of whole blood, obviating venipuncture and expensive equipment for serum separation, and performing with high sensitivity and specificity.

The point-of-care testing for Toxoplasma infection using whole-blood as disclosed herein meets all of the WHO criteria for POC testing. It is an Affordable, Sensitive, Specific, User friendly, Rapid/robust, Equipment free, and Deliverable to end users (A.S.S.U.R.E.D.).

Adoption of this whole-blood POC test would reduce costs further substantially and require significantly less infrastructure than conventional testing. Value also arises from bringing pregnant women into care, ensuring screening for other preventable and treatable conditions, particularly in developing countries [6]. This whole blood POC test was not used to distinguish seropositivity for IgG and IgM, and therefore did not distinguish acute from chronic infection. Confirmatory testing and guidance concerning additional testing and treatment will benefit patients and their physicians. In countries with high seroprevalence, this benefit would be particularly important. This POC test demonstrated superb diagnostic performance in countries in the developed and developing world, with genetically distinct patients and parasites.

### Advantages:

True point-of-care test, useful in a standard obstetrician's office and less invasive than conventional serologic testing (requiring materials to perform venipuncture, electricity and a centrifuge for serum separation, and skilled technicians). POC testing has potential to significantly expand access to screening during gestation for this serious, potentially fatal infection, with spillover benefit in facilitating screening programs for other congenital infections and improvements in maternal fetal health and well-being and care. The high performance of this whole-blood POC test as demonstrated herein, and the test's strong functionality at the point-of-care, which has not been previously demonstrated, provides the proof of principle of its potential utility and widespread applicability in clinical settings. This new test also fulfills the World Health Organization criteria for the ideal POC test (Affordable, Sensitive, Specific, User-friendly, Rapid/robust, Equipment-free, and Deliverable to users).
Performance and interpretation of test takes less than two minutes of operator time, with results available to be interpreted within 20-30 minutes, facilitating appropriate clinical intervention so that it is ideal for following those who are seronegative to detect seroconversion
There is no need for expensive equipment/electricity, decreasing cost and enhancing utility, especially in developing countries

### REFERENCES

1. McLeod R, Wheeler K, Levigne P, Boyer K. Toxoplasma gondii. In: Read J S, Schleiss M R, eds. Congenital and Perinatal Infections. Oxford University Press, 2018: 223-239.
2. McLeod R, Lykins J, Noble A G, et al. Management of Congenital Toxoplasmosis. Curr Pediatr Rep 2014; 2:166-194.
3. Stillwaggon E, Carrier C S, Sautter M, McLeod R. Maternal Serologic Screening to Prevent Congenital Toxoplasmosis: A Decision-Analytic Economic Model. PLoS Negl Trop Dis 2011; 5:e1333.
4. McLeod R, Boyer K, Karrison T, et al. Outcome of Treatment for Congenital Toxoplasmosis, 1981-2004: The National Collaborative Chicago-Based, Congenital Toxoplasmosis Study. Clin Infect Dis 2006; 42:1383-1394.
5. Wallon M, Peyron F, Cornu C, et al. Congenital Toxoplasma Infection: Monthly Prenatal Screening Decreases Transmission Rate and Improves Clinical Outcome at Age 3 Years. Clin Infect Dis 2013; 56:1223-1231.
6. Begeman I J, Lykins J, Zhou Y, et al. Point-of-care testing for Toxoplasma gondii IgG/IgM using Toxoplasma ICT IgG-IgM test with sera from the United States and implications for developing countries. PLoS Negl Trop Dis 2017; 11:e0005670.
7. Chapey E, Wallon M, Peyron F. Evaluation of the LDBIO point of care test for the combined detection of toxoplasmic IgG and IgM. Clin Chim Acta 2017; 464:200-201.
8. Maldonado Y A, Read J S, Diseases C on I. Diagnosis, Treatment, and Prevention of Congenital Toxoplasmosis in the United States. Pediatrics 2017; :e20163860.
9. Prusa A-R, Kasper D C, Sawers L, Walter E, Hayde M, Stillwaggon E. Congenital toxoplasmosis in Austria: Prenatal screening for prevention is cost-saving. PLoS Negl Trop Dis 2017; 11:e0005648.
10. Mahinc C, Flori P, Delaunay E, et al. Evaluation of a new ICT test (LDBIO Diagnostics) to detect toxoplasma IgG and IgM: comparison with the routine Architect technique. J Clin Microbiol 2017; :JCM.01106-17.

1. A method for diagnosing a Toxoplasma infection in a subject at risk of a Toxoplasma infection, comprising contacting a whole blood sample from the subject to a sample well of a lateral flow immunochromatography device to diagnose a likelihood of the subject having a Toxoplasma infection.

2. The method of claim 1, wherein the lateral flow immunochromatography device comprises:
(a) providing a cassette, wherein the cassette comprises:
(i) a nitrocellulose strip comprising a Toxoplasma antigen immobilized on a test band; and
(ii) a conjugate pad in fluid communication with the nitrocellulose strip impregnated with test latex particles coupled with a Toxoplasma antigen; and
(iii) a sample well in fluid communication with the conjugate pad;
(b) adding the whole blood sample to the sample well to initiate migration of the whole blood and the test latex particles along the conjugate pad, under conditions to promote binding of anti-Toxoplasma antibodies in the whole blood sample to the Toxoplasma antigen on the test latex particles to form positive complexes; and
(c) detecting an interaction between the positive complexes and the test band.

3. The method of claim 1, wherein approximately 30 μL of the whole blood sample is added to the sample well.

4. The method of claim 2, wherein the nitrocellulose strip further comprises control latex particles that are optically distinguishable from the test latex particles, and wherein the conjugate pad of further comprises control antigens immobilized on a control band.

5. The method of claim 2, wherein the test latex particles are black and the control latex particles are blue.

6. The method of claim 2, wherein the control latex particles are impregnated with goat anti-rabbit antibodies, and wherein the control antigens comprise rabbit gamma immunoglobulins.

7. The method of claim 2, comprising dispensing an eluting solution to the sample well, subsequent to the whole blood sample, and allowing migration of the whole blood sample and the eluting solution for 20-30 minutes before reading the test band and the control band of the device.

8. The method of claim 2, wherein the anti-Toxoplasma antibodies in the whole blood sample are IgG and/or IgM antibodies.

9. The method of claim 1, wherein the Toxoplasma infection is the result of an infection by Toxoplasma gondii.

10. The method of claim 1, wherein the subject is a pregnant human or a human suspected of being pregnant.

11. The method of claim 1, further comprising treating those subjects that are identified as likely to have a Toxoplasma infection with a therapeutic for treating Toxoplasma infection.

12. The method of claim 11, wherein the therapeutic is selected from the group consisting of: pyrimethamine, sulfadiazine, sulfapyrizine, sulfamethazine, sulfamerizine, azithromycin, clarithromycin, atovaquone, dapsone, cotrimoxazole, and combinations thereof.

13. A kit comprising a lateral flow immunochromatography device for diagnosing a Toxoplasma infection in a subject wherein the kit comprises:
(a) the lateral flow immunochromatography device, wherein the device comprises a cassette comprising:
(i) a nitrocellulose strip on which are immobilized a Toxoplasma antigen in a test band and a control antigen in a control band;
(ii) a conjugate pad in fluid communication with the nitrocellulose strip impregnated with test latex particles coupled with a Toxoplasma antigen and control latex particles coupled with a control antigen; and
(iii) a sample well in fluid communication with the conjugate pad;
(b) eluting buffer.

14. The kit of claim 13, wherein the kit comprises a timer.

15. The kit of claim 13, wherein the eluting buffer is in a 3 mL dropper bottle.

16. The kit of claim 13, wherein the kit comprises:
(c) 60 lateral flow immunochromatography devices, wherein there are 6 bags, and each bag contain 10 devices and a desiccant packet;
(d) 3 dropper bottles with the eluting buffer, where each dropper bottle comprises 3 mL of eluting buffer; and
(e) instructions for use.

17. The kit of claim 13, wherein the kit comprises:
(f) lancets for fingertip whole blood sampling; and
(g) capillary tubes for collecting the whole blood sample.
